(19) United States
(12) Patent Application Publication
(10) Pub. No.: US 2025/0201368 A1
(43) Pub. Date: Jun. 19, 2025

(54) MULTISCALE GRAPH-BASED ANALYSIS AND VISUALIZATION OF CLINICAL DATA

(71) Applicant: INTEGO GROUP, LLC, Maitland, FL (US)

(72) Inventors: Sergey Glushakov, Orlando, FL (US); Kostiantyn Drach, Barcelona (ES); Viktoriia Shevtsova, Kharkiv (UA); Iryna Kotenko, Kharkiv (UA); Lyudmyla Polyakova, Kharkiv (UA); Oleksandr Leonov, Kharkiv (UA)

(21) Appl. No.: 19/070,724

(22) Filed: Mar. 5, 2025

Related U.S. Application Data
(63) Continuation-in-part of application No. 18/380,213, filed on Oct. 16, 2023, now Pat. No. 12,278,003.
Application No. 18/380,213 is a continuation-in-part of application No. 17/380,472, filed on Jul. 20, 2021, now Pat. No. 11,789,970.
Application No. 17/380,472 is a continuation-in-part of application No. 16/147,640, filed on Sep. 29, 2018, now Pat. No. 11,069,447.

(51) Int. Cl. G16H 15/00; G16H 10/20

(52) U.S. Cl. CPC G16H 15/00; G16H 10/20

(57) ABSTRACT

Methods and systems for multiscale analysis and visualization of clinical data are provided. An example method includes receiving vectors of outcomes of trial subjects, generating a plurality of metric graphs including first nodes corresponding to the vectors of outcomes and selectively connected based on a first criterion, selecting, from the plurality of metric graphs and based on a second criterion, an optimal graph, generating, based on the optimal graph and a resolution parameter, a clustered graph including second nodes corresponding to groups of the first nodes, the second nodes being selectively connected based on a third criteria, generating a first layout of the clustered graph, the first layout including a two-dimensional (2D) representation of the clustered graph, generating, partially based on the first layout, a second layout of the optimal graph, the second layout including a 2D representation of the optimal graph, and displaying the second layout.

### CROSS-REFERENCE TO RELATED APPLICATIONS

This application is a Continuation-in-part of U.S. patent application Ser. No. 18/380,213, entitled “Automatic Selection of Optimal Graphs with Robust Geometric Properties in Graph-based Discovery of Geometry of Clinical Data,” filed on Oct. 16, 2023, which in turn is a Continuation-in-part of U.S. patent application Ser. No. 17/380,472, entitled “Graph-Based Discovery of Geometry of Clinical Data to Reveal Communities Of Clinical Trial Subjects,” filed on Jul. 20, 2021, which in turn a Continuation-in-part of U.S. patent application Ser. No. 16/147,640, entitled “Systems and Methods for Topology-Based Clinical Data Mining,” filed on Sep. 29, 2018. The subject matter of aforementioned applications is incorporated herein by reference in its entirety for all purposes.

### TECHNICAL FIELD

This disclosure generally relates to clinical data processing. More specifically, this disclosure relates to systems and methods for multiscale graph-based analysis and visualization of clinical data.

### BACKGROUND

Clinical trials are designed to assess the safety and efficacy of biomedical or behavioral interventions. Typically, investigators use only a small portion of the data collected during these trials to demonstrate a medical intervention's safety and efficacy. However, clinical trials generate substantial amounts of data that can later be analyzed to identify unexpected factors influencing outcomes and to develop new hypotheses.

Performing a comprehensive analysis of a clinical trial dataset is challenging. Most approaches to mining clinical data focus on univariate relationships between a specific outcome and a few predictive variables. Yet, there is a lack of data integration and visualization tools that provide a holistic understanding of the entire dataset. Focusing on a single outcome in isolation from other factors may lead to an incomplete—or even misleading—perspective on complex scenarios. Standard biostatistical methods can confirm or refute hypotheses generated by investigators, but they rely heavily on the researcher's ability to formulate robust hypotheses. In clinical trial datasets, the sheer number of potential hypotheses makes it very difficult to select the most relevant ones.

Graph-based analysis and visualization of clinical trial datasets is a promising approach. However, constructing these graphs from clinical trial data and presenting them in a user-friendly manner remain challenging.

### SUMMARY

This summary is provided to introduce a selection of concepts in a simplified form that are further described in the Detailed Description below. This summary is not intended to identify key features or essential features of the claimed subject matter, nor is it intended to be used as an aid in determining the scope of the claimed subject matter.

According to one example embodiment of the present disclosure, a method for multiscale graph-based analysis and visualization of clinical data is provided. The method may include receiving vectors of outcomes of trial subjects and generating, based on the vectors of outcomes, a plurality of metric graphs. A metric graph of the plurality of metric graphs may include first nodes corresponding to the vectors of outcomes. The first nodes can be selectively connected based on a first criterion. The method may also include selecting, from the plurality of metric graphs and based on a second criterion, an optimal graph and generating, based on the optimal graph, a clustered graph. The clustered graph may include second nodes corresponding to groups of the first nodes. The second nodes can be selectively connected based on a third criteria. The method may include generating a first layout of the clustered graph. The first layout may include a two-dimensional (2D) representation of the clustered graph. The method may also include generating, partially based on the first layout, a second layout of the optimal graph and displaying the second layout. The second layout may include a 2D representation of the optimal graph.

The method may include, prior to the generating the clustered graph, receiving a resolution parameter and determining the groups of the first nodes based on the resolution parameter. The resolution parameter can be received from a user via a user interface. The determination of the groups may include projecting the first nodes onto a set of domains and determining a tree including the first nodes having projections in a domain of the set of domains, such that the tree has a minimum sum of distances between the first nodes connected in the tree. The determination of the groups may further include determining, based on the tree and the resolution parameter, subtrees of the tree and forming the groups based on the subtrees. A number of subtrees can be selected based on the product of the resolution parameter and the number of the first nodes having projections in the domain of the set of domains.

The second layout can be determined by an iteration procedure starting with an approximate layout. The approximate layout can be determined based on the first layout of the clustered graph. The method may also include displaying the first layout of the clustered graph synchronously with the second layout of the optimal graph.

The method may include determining that the optimal graph includes a first subgraph and a second subgraph, wherein nodes of the first subgraph disconnected from further nodes of the second subgraph, adding a connection between a node of the first subgraph and a further node of the second subgraph, and displaying the connection using one of the following: a line and a collection of lines. At least one characteristic of the line can differ from characteristics of a further line, where the further line is used to display one of the following: a connection between the nodes of the first subgraph and a connection between the nodes of the second subgraph.

According to another embodiment, a system for multiscale graph-based analysis and visualization of clinical data is provided. The system may include at least one processor and a memory storing processor-executable codes, wherein the processor can be configured to implement the operations of the above-mentioned method for multiscale graph-based analysis and visualization of clinical data.

According to yet another aspect of the disclosure, there is provided a non-transitory processor-readable medium, which stores processor-readable instructions. When the processor-readable instructions are executed by a processor, they cause the processor to implement the above-mentioned method for multiscale graph-based analysis and visualization of clinical data.

Other example embodiments of the disclosure and aspects will become apparent from the following description taken in conjunction with the following drawings.

### BRIEF DESCRIPTION OF DRAWINGS

Exemplary embodiments are illustrated by way of example and not limitation in the figures of the accompanying drawings, in which like references indicate similar elements.

FIG. 1 is a block diagram showing an example architecture, wherein methods for multiscale graph-based analysis and visualization of clinical data can be implemented.

FIG. 2 is a block diagram showing a system for multiscale graph-based analysis and visualization of clinical data, according to one example embodiment.

FIG. 3 is a schematic diagram showing data points, projections of data points, and overlapping domains covering the projections, according to an example embodiment.

FIG. 4 illustrates minimum spanning trees for overlapping domains, according to an example embodiment.

FIG. 5 illustrates an example plot of the distribution of the lengths of the edges, probability of deviation of lengths of edges from the mean, and regions for outliers, according to an example embodiment.

FIG. 6 is a schematic diagram illustrating minimum spanning trees for overlapping domains and corresponding plots of distributions of the lengths of the edges in the minimum spanning trees, according to an example embodiment.

FIG. 7 is a schematic diagram illustrating plots of metrics corresponding to iterations of an example method for determining communities in a metric graph, according to an example embodiment.

FIG. 8 is a schematic diagram showing an example metric graph and corresponding clustered graph, according to an example embodiment.

FIG. 9 is a schematic diagram showing scaled clustered graphs corresponding to the metric graph of FIG. 8, according to an example embodiment.

FIG. 10 is a schematic diagram showing an example metric graph, communities, and corresponding community-clustered graph, according to an example embodiment.

FIG. 11 is a schematic diagram showing different layouts of metric graphs, according to some example embodiments.

FIG. 12 is a schematic diagram illustrating a layout of a metric graph and two synchronized layouts of clustered graphs corresponding to the metric graph, according to an example embodiment.

FIG. 13 is a schematic diagram illustrating a layout of a metric graph and two synchronized layouts of clustered graphs corresponding to the metric graph, according to another example embodiment.

FIG. 14 is a schematic diagram showing layouts of metric graphs with application of different conformal mappings, according to some example embodiment.

FIG. 15 is a schematic diagram showing layouts of metric graphs with application of different conformal mappings, according to another example embodiment.

FIG. 16 is a schematic diagram depicting two layouts of a metric graph, according to an example embodiment.

FIG. 17 is a process flow diagram showing a method for multiscale graph-based analysis and visualization of clinical data, according to an example embodiment.

FIG. 18 is a high-level block diagram illustrating an example computer system, within which a set of instructions for causing the machine to perform any one or more of the methodologies discussed herein can be executed.

### DETAILED DESCRIPTION

The following detailed description of embodiments includes references to the accompanying drawings, which form a part of the detailed description. Approaches described in this section are not prior art to the claims and are not admitted to be prior art by inclusion in this section. The drawings show illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical, and operational changes can be made without departing from the scope of what is claimed. The following detailed description is, therefore, not to be taken in a limiting sense, and the scope is defined by the appended claims and their equivalents.

Generally, the embodiments of this disclosure are concerned with methods for multiscale graph-based analysis and visualization of clinical data. The methods described herein can be implemented by hardware modules, software modules, or a combination of both. The methods can also be embodied in computer-readable instructions stored on computer-readable media. As should be evident from the following description, the methods and systems of this disclosure allow mining for hidden patterns in clinical datasets. Embodiments of the present disclosure may also provide an interactive visualization application allowing researchers to explore groups of trial subjects with similar outcomes and perform statistical analysis of predictors of trial subjects within the groups.

The embodiments will now be presented with reference to the accompanying drawings. These embodiments are described and illustrated by various modules, blocks, components, circuits, steps, operations, processes, algorithms, and the like, collectively referred to as “components” for simplicity. These components may be implemented using electronic hardware, computer software, or any combination thereof. Whether such components are implemented as hardware or software depends upon the particular application and design constraints imposed on the overall system. By way of example, a component, or any portion of a component, or any combination of components may be implemented with a “processing system” that includes one or more processors. Examples of processors include microprocessors, microcontrollers, Central Processing Units (CPUs), digital signal processors (DSPs), field programmable gate arrays (FPGAs), programmable logic devices (PLDs), state machines, gated logic, discrete hardware circuits, and other suitable hardware configured to perform various functions described throughout this disclosure. One or more processors in the processing system may execute software, firmware, or middleware (collectively referred to as “software”). The term “software” shall be construed broadly to mean instructions, instruction sets, code, code segments, program code, programs, subprograms, software components, applications, software applications, software packages, routines, subroutines, objects, executables, threads of execution, procedures, functions, and the like, whether referred to as software, firmware, middleware, microcode, hardware description language, or otherwise. The software may be stored on or encoded as one or more instructions or code on a non-transitory computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), compact disk ROM (CD-ROM) or other optical disk storage, magnetic disk storage, solid state memory, or any other data storage devices, combinations of the aforementioned types of computer-readable media, or any other medium that can be used to store computer executable code in the form of instructions or data structures that can be accessed by a computer.

For purposes of this document, the terms “or” and “and” shall mean “and/or” unless stated otherwise or clearly intended otherwise by the context of their use. The term “a” shall mean “one or more” unless stated otherwise or where the use of “one or more” is clearly inappropriate. The terms “comprise,” “comprising,” “include,” and “including” are interchangeable and not intended to be limiting. For example, the term “including” shall be interpreted to mean “including, but not limited to.”

The term “module” shall be construed to mean a hardware device, software, or a combination of both. For example, a hardware-based module can use either one or more microprocessors, application-specific integrated circuits (ASICs), programmable logic devices, transistor-based circuits, or various combinations thereof. Software-based modules can constitute computer programs, computer program procedures, computer program functions, and the like. In addition, a module of a system can be implemented by a computer or server, or by multiple computers or servers connected into a network. Hardware or software implementations can depend on particular system implementation and constraints. For example, a communication module may include a radio modem, Ethernet module, network interface, communication port, or circuit terminals. In other embodiments, a communication module may include software, software procedure, or software-based function configured to receive and transmit data by a hardware device, such as a processor. Other implementations of communication module can involve programmable and non-programmable microcontrollers, processors, circuits, computing devices, servers, and the like.

The terms “topological data map”, “data map”, and “graph” shall be construed to mean the same and refer to the visual representation of individual trial subjects or groups of trial subjects by nodes connected with edges.

The terms “trial subject”, “study subject”, “human subject”, and “subject” shall be construed to mean the same and refer to an individual who is the source of data for a research investigator through intervention or interaction with the individual or from individually identifiable information. Such individuals can include healthy humans or patients.

Referring now to the drawings, various embodiments are described in which like reference numerals represent like parts and assemblies throughout the several views. It should be noted that the reference to various embodiments does not limit the scope of the claims attached hereto. Additionally, any examples outlined in this specification are not intended to be limiting and merely set forth some of the many possible embodiments for the appended claims.

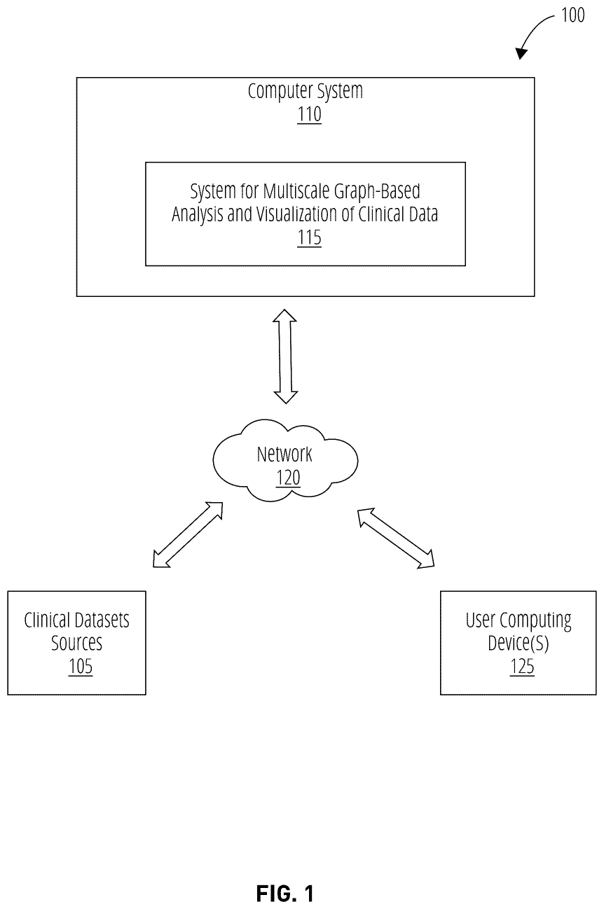

FIG. 1 is a block diagram showing an example architecture 100 suitable for implementing methods for multiscale graph-based analysis and visualization of clinical data, according to some example embodiments. The architecture 100 may include one or more clinical datasets sources 105, a computing system 110, one or more user computing device(s) 125, and a network 120.

The clinical datasets sources 105 may include server(s) configured to store and provide access to clinical datasets. The clinical datasets can be formatted according to a standard format (for example, a Clinical Data Interchange Standards Consortium (CDISC) format, a Study Data Tabulation Model (SDTM) format, an analysis data model (ADaM) format, and the like).

The computing system 110 may include a standalone server or cloud-based computing resource(s). The standalone server or the cloud-based computing resource(s) can be shared by multiple users. The cloud-based computing resource(s) can include hardware and software available at a remote location and accessible over the network 120. The cloud-based computing resource(s) can be dynamically re-allocated based on demand. The cloud-based computing resources may include one or more server farms/clusters including a collection of computer servers which can be co-located with network switches and/or routers. The computing system 110 may include a system 115 for multiscale graph-based analysis and visualization of clinical data.

The user computing device(s) 125 may include a personal computer, a laptop computer, tablet computer, smartphone, server computer, network storage computer, or any other computing device comprising at least networking and data processing capabilities.

The network 120 may include any wired, wireless, or optical networks including, for example, the Internet, intranet, local area network (LAN), Personal Area Network (PAN), Wide Area Network (WAN), Virtual Private Network (VPN), cellular phone networks (e.g., Global System for Mobile (GSM) communications network, packet switching communications network, circuit switching communications network), Bluetooth radio, Ethernet network, an IEEE 802.11-based radio frequency network, a Frame Relay network, Internet Protocol (IP) communications network, or any other data communication network utilizing physical layers, link layer capability, a network layer to carry data packets, or any combinations of the above-listed data networks.

Users of the user computing device(s) 125 may access the system 115 using one or more applications of the user computing device(s) 125, for example a web browser, via the network 120. The users may configure the system 115 by selecting clinical datasets and indicating parameters for construction of graphs representing the data in clinical datasets. The system 115 may be further configured to display a graphical representation of the graphs and provide users with a means for selecting groups of trial subjects using the graphical representation.

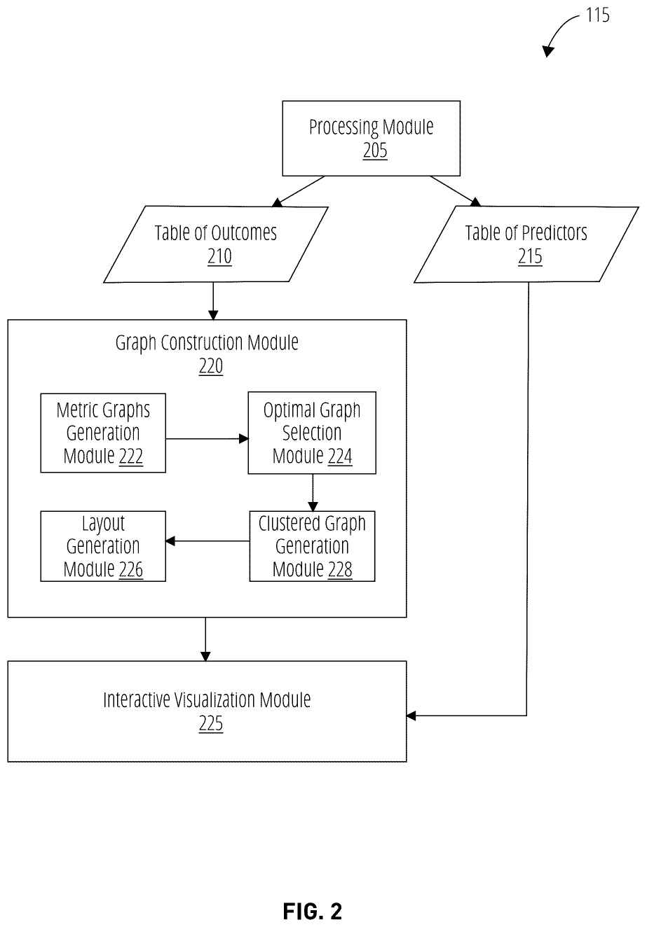

FIG. 2 is a block diagram showing an example system 115 for multiscale graph-based analysis and visualization of clinical data, according to some example embodiments. The system 115 may include a processing module 205, a graph construction module 220, and an interactive visualization module 225. The graph construction module 220 may include a metric graphs generation module 222, an optimal graph selection module 224, a layout generation module 226, and a clustered graph generation module 228.

The processing module 205 may be configured to transform original clinical datasets into a table of outcomes 210 and a table of predictors 215. The table of outcomes 210 may include rows representing trial subjects and columns representing outcomes. The outcomes (also known as response variables) may include biomarkers, results of measurement of vital signs, results of physiological measurements, and questionnaire items recorded during medical treatment of trial subjects. Examples of the outcomes are levels of serum creatinine, blood urea nitrogen, and neutrophil gelatinase-associated lipocalin as a means of evaluating kidney function, absolute or percentage change in the tumor size over the course of study, quality of life score and so forth. The outcome may include questionnaire item to assess trial subject's general health or quality of life, and the like.

The table of predictors 215 may include rows corresponding to the trial subjects and predictors associated with the trial subjects. The predictors may include, for example, demographic attributes, such as sex, age, ethnicity, and residence. The predictors may also include medical history attributes and medical interventions attributes.

The clinical datasets can include quantitative data, binary data, or categorical data. The processing module 205 may transform the categorical data into numerical values. For example, an “emotional level” can be represented by numbers of 1 to 7. One of the main problems of clinical datasets is missing values. Therefore, the processing module 205 can be configured to fill in missing values for outcomes in table of predictors 215. The processing module 205 can also be configured to combine one or more variables of the clinical data to synthetic variables to aggregate more data for an analysis.

The processing module 205 can be further configured to normalize the values of outcomes to facilitate measurement of distances between data points to find similarities in the clinical datasets. Data points may include row vectors {x=(x₁, x₂, . . . , xₙ)}, wherein each vector corresponds to a single trial subject x, and xᵢ denotes the i-th outcome for the trial subject x.

The metric graphs generation module 222 can be configured to generate, based on the table of outcomes 210, a plurality of metric graphs (also referred to as “topological data map” or “data map”). In each metric graph of the plurality of metric graphs, a single node corresponds to an individual trial subject. If two nodes represent similar trial subjects (in terms of pre-defined outcomes), they are connected with an edge.

In some embodiments, to determine whether two trial subjects are similar, a distance between two data points representing the two trial subjects can be calculated according to a distance function. If the distance does not exceed a distance threshold, then the two nodes (representing the two trial subjects) are connected with an edge.

The construction of a metric graph may depend on a selection of outcomes to be considered when calculating the distance, a distance function to calculate the distance, and a distance threshold. By changing the selection of outcomes, the distance function and the distance threshold, substantial number of metric graphs can be generated and included into the plurality of metric graphs.

If the data points represent purely quantitative data, a Euclidean distance, a normalized Euclidean distance, a Manhattan distance, and a Minkowski distance can be used to calculate distances between the data points. A Hamming distance can be used to calculate a distance if the data points represent purely categorical data. If several outcomes of different types (quantitative, binary, categorical) are combined in the table of outcomes, then the data points represent mixed data (quantitative data and categorical data). When the data points represent mixed data, a more general measure of a distance, such as the Gower distance, can be used.

In other embodiments, prior to construction of the metric graphs, the data points {x=(x₁, x₂, . . . , xₙ)} can be divided into overlapping subsets. During the construction of the metric graphs, a distance function and distance threshold can be selected independently for each of the overlapping subsets. To obtain the overlapping subsets, each data point {x=(x₁, x₂, . . . , xₙ)} is mapped by a projection rule (referred to as a “projection”) to the unique point in the set of points {p=(p₁, p₂, . . . , pₘ)} (referred to as “the values of the projection” or “projection values”). The projections can be one-dimensional (corresponds to m=1) or multidimensional (corresponding to m>1). The values of the projections can be further divided into overlapping domains. The data points corresponding to one of the overlapping domains can be further collected into one of the overlapping subsets.

The graph construction module 220 can be further configured to select an optimal graph (also referred herein as to a graph of interest) from the metric graphs. The optimal graph can be determined as the most representative metric and most stable graph. To determine the most representative and most stable graph, the graph construction module 220 can calculate values of one or more objective functions of the metric graphs. The objective functions map a set of metric graphs to real numbers. The metric graph having the highest value of one of the objective functions can be selected as the optimal graph. In some embodiments, the objective function may include a projection-driven modularity of the metric graphs. According to some embodiments of the present disclosure, a projection-driven modularity of a metric graph can be defined as a value that measures a difference between the metric graph and a random graph. The difference can be measured within each individual subgraph comprising nodes whose projection values fall into the same domain among the overlapping domains that were used to construct the metric graphs of the plurality of metric graphs.

The graph construction module 220 can be further configured to generate a clustered graph from the graph of interest. In the optimal graph, which is a metric graph, every node corresponds to a single trial subject while two nodes representing trial subjects (in terms of pre-defined outcomes) are connected with an edge. Thus, the clustered graph may represent a compressed version of the optimal graph. The compressed version can be obtained using one or more algorithms for clustering of nodes of graphs or community detection in graphs. Unlike the optimal graph (which is a metric graph), each node in the clustered graph corresponds to a group of trial subjects. The groups corresponding to different nodes of the clustered graph do not overlap, ensuring that no trial subject belongs to more than one node in the clustered graph.

The clustering of a metric graph (for example optimal graph) can be also based on a modularity of groups of nodes in the metric graph. A cluster can be determined as a group of nodes of the metric graph, wherein the number of edges between nodes within the group is significantly more than the expected number of edges if the edges were distributed randomly within the graph. The modularity reflects a concentration of edges within the cluster in comparison to a random distribution of edges between all nodes in the metric graph according to a statistical model.

The graph construction module 220 can be further configured to generate layouts of the optimal graph in forms of a metric graph and the clustered graph. The layouts can be further used in graphical presentations of the optimal graph. Layout of the nodes of the clustered graph can be visually aligned with a layout of corresponding groups of nodes of the metric graph.

The interactive visualization module 225 can be configured to display a graphical representation of the optimal graph. A user may perform a visual exploration of the optimal graph to discover structural features. In some embodiments, the interactive visualization module 225 may provide a web-based interface for the user. The web-based interface may provide basic operations for visual exploration. Based on a user selection, interactive visualization module 225 may display the graphical representation of the optimal graph in the form of a metric graph or a clustered graph corresponding to optimal graph. The interactive visualization module 225 may allow zooming in, zooming out, and panning of the graphical representation. The interactive visualization module 225 may provide an additional information for each node using a pop-up window when a user positions a mouse over the node. The interactive visualization module 225 may provide a means for selection of groups of nodes. For example, interactive visualization module 225 may allow the user to select one or two groups of nodes of the optimal graph. The selected groups can be further used in statistical analysis of predictors associated with trial subjects in the selected groups.

The interactive visualization module 225 may be configured to color the nodes in the graphical representation of the optimal graph. The color of a node can be based on the value of one or more predictors or outcomes of a trial subject that the node represents. The color of a node can be based on a projection value of a data point. A user may re-color the nodes in the graphical representation by selecting a specific outcome or a specific predictor. The color of the nodes may highlight differences between a subgroup of trial subjects represented by a given region of the graphical representation and the rest of the trial subjects participating in a clinical trial, and, thereby, highlight patterns in the clinical datasets. The color of the nodes may also help the user to identify groups of trial subjects to be selected for statistical analysis.

The interactive visualization module 225 may be further configured to perform a statistical analysis of predictors related to trial subjects in the selected groups. In some embodiments, the user can select a region of the optimal graph to specify a group of trial subjects. Then statistical analysis can be performed to find predictors that explain why these trial subjects are combined into a group. After running statistical tests, a table of predictors with their corresponding p-values can be calculated to determine if a distribution of values of the predictors for the selected group of trial subjects is different from a distribution of values of the predictors of the rest of the trial subjects participating in the clinical study.

In some embodiments, the user can select a first region and a second region in the optimal graph, and, thus, select a first group of trial subjects and a second group of trial subjects. The interactive visualization module 225 may further perform calculations of p-values for the statistical tests to determine if a distribution of values of the predictor of the first group of trial subjects is different from a distribution of values of the predictor of the second group of trial subjects.

The interactive visualization module 225 may be also configured to perform an automatic search to highlight a group of related trial subjects in the optimal graph. The automatic search can be performed in addition to the visual inspection of the optimal graph that can be performed by a user. The automatic search can be carried out using machine learning algorithms for automated discovery of groups of trial subjects with common features and similarities.

The interactive visualization module 225 can be configured to allow a user to export data for the selected groups of trial subjects and generate one or more reports. The reports may include details of the statistical analysis in the form of a table and charts. The reports can be generated in a portable data format. The data concerning the selected groups of trial subjects may include a table of outcomes and predictors of the trial subjects in the selected group. The data can be exported in comma-separated values or other formats that are acceptable by external statistical analysis platforms. A user may use the exported data to determine other explanatory variables (predictors) that may be responsible for the similarities of responses observed within each selected group of the trial subjects who participated in clinical trial. An additional statistical analysis of the exported data can be performed using SAS™, R, or another data analytics platform.

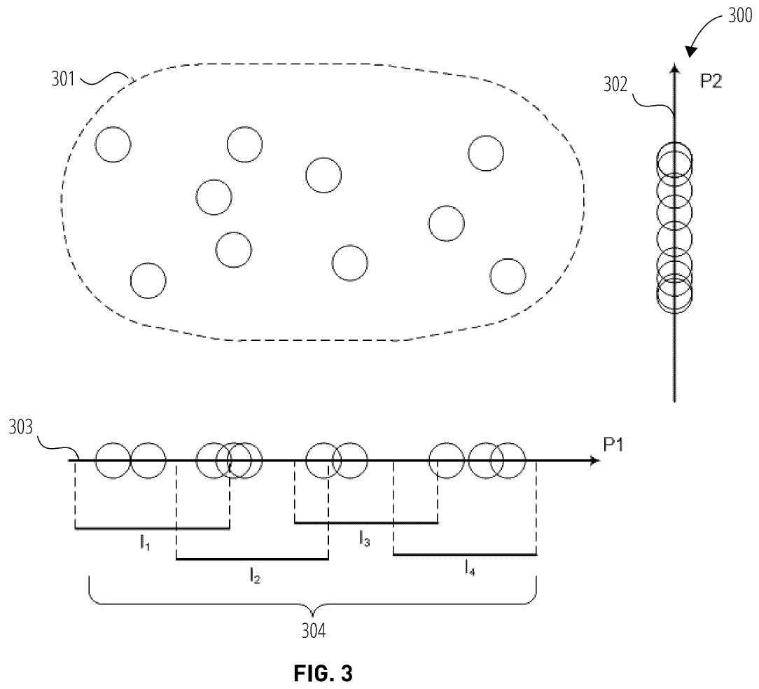

FIG. 3 is schematic diagram 300 showing data points 301, projections 302, projections 303, and overlapping domains 304, according to an example embodiment. The overlapping domains 304 (Iₜ) may be constructed by calculating range for each of the projections 302 as an interval between maximal and minimum component pᵢ in {(p₁(k), p₂(k), . . . , pₘ(k))}. Then the range can be covered by qᵢ overlapping domains 304, lₗ, l=1, . . . , qᵢ and selecting a percentage of overlapping. The interval Iₗ overlaps the interval Ij with percentage oₗⱼ of overlapping if the length of interval Iₗ∩Iⱼ is oₗⱼ percent of the length of interval Ij. It should be noted that, in general, oₗⱼ≠oⱼₗ. In some embodiments, the overlap parameter can be the same for all pairs of overlapping intervals. In certain embodiments, the overlap parameter is 50 percent. Larger overlap parameters result in obtaining the metric graphs having more connected nodes, which is preferable. The number of overlapping domains (qᵢ) can be selected such that the number of projections of data points from the source dataset falling into a single overlapping domain (interval) is within a predetermined limit. In certain embodiments, the predetermined limit is 1000.

A uniform covering can be used if the distribution of the projections is close to a uniform distribution. The uniform covering is a covering with the entire range being covered by intervals of equal length. In this case, each overlapping interval may contain a different number of points. A balanced covering can be used if the distribution of projection values is extremely uneven. A balanced covering is a covering with overlapping intervals containing an approximately equal number of projections.

In both cases, the boundaries of overlapping intervals can be determined by the number of intervals and percentage of overlapping. The selection of the number of the intervals and the percentage of overlapping for uniform covering may result in unambiguous coverage. The selection of the number of the intervals and the percentage of overlapping for the balanced covering may result in different coverings. However, using different balanced coverings does not affect the structure of the graph.

Multidimensional overlapping domains can be obtained as the Cartesian product of two and more one-dimensional coverages. A (k₁, k₂, . . . kₘ) multidimensional domain may include points that lie simultaneously in the k₁-th domain of the first one-dimensional coverage, the k₂-th of the second one-dimensional coverage, and so forth. In other embodiments, multidimensional overlapping domains can be different from the Cartesian products of one-dimensional domains.

In some embodiments, metric graphs generation module 222 (shown in FIG. 2), may generate a plurality of metric graphs using the following approach described in the U.S. patent application Ser. No. 17/380,472, entitled “Graph-Based Discovery of Geometry of Clinical Data to Reveal Communities Of Clinical Trial Subjects,” now the U.S. Pat. No. 11,789,970, which is incorporated herein by reference in its entirety for all purposes.

To construct a metric graph, a projection rule from a set of projection rules can be selected. The selected projection rule can be applied to data points 301 to obtain projections of the vectors of outcomes. For each of a first node and a second node from the set of nodes corresponding, the metric graphs generation module 222 can determine that a first projection and a second projection of the projections satisfy similar criteria, where the first projection corresponding to the first node and the second projection corresponding to the second node. Then based on the determination that the first projection and the second projection satisfy the similarity criteria, metric graphs generation module 222 selectively connect the first node and the second node. By selecting different projections rules from the set of projection rules, different metric graphs can be constructed and included into the plurality of metric graphs. In some embodiments, the determination that the first projection and the second projection satisfy the similar criteria may include determining that the first projection and the second projection are located within a same domain in a set of overlapping domains.

In some other embodiments, the determination that the first projection and the second projection satisfy the similarity criteria may also include the following: 1) by varying a level of granularity, constructing a tree of clusters of the projections belonging to the same overlapping domain; and 2) determining that the first projection and the second projection belong to a same cluster from the tree of clusters, wherein the same cluster obtained with an optimal level of granularity obtained for the same domain. Optimal level of granularity can be selected such that a number of clusters corresponding to the optimal level of granularity is less than a half of a total number of the projections in the same domain.

According to some example embodiments of this disclosure, clusters within the same overlapping domain can be determined based on a cutoff threshold. A cluster may include those projections from the overlapping domain that are within a distance less than the cutoff threshold from a point in that overlapping domain. The cutoff threshold can be calculated using the following approach.

First, for the projections in an overlapping domain, the minimum spanning tree is constructed. The minimum spanning tree can be constructed as a spanning tree of the complete graph on the set of nodes corresponding to the projections within the overlapping domain such that the total length (or weight) of edges of the spanning tree is minimal. If several minimum spanning trees can be constructed for an overlapping domain, any of them can be selected for further calculation of the cutoff threshold.

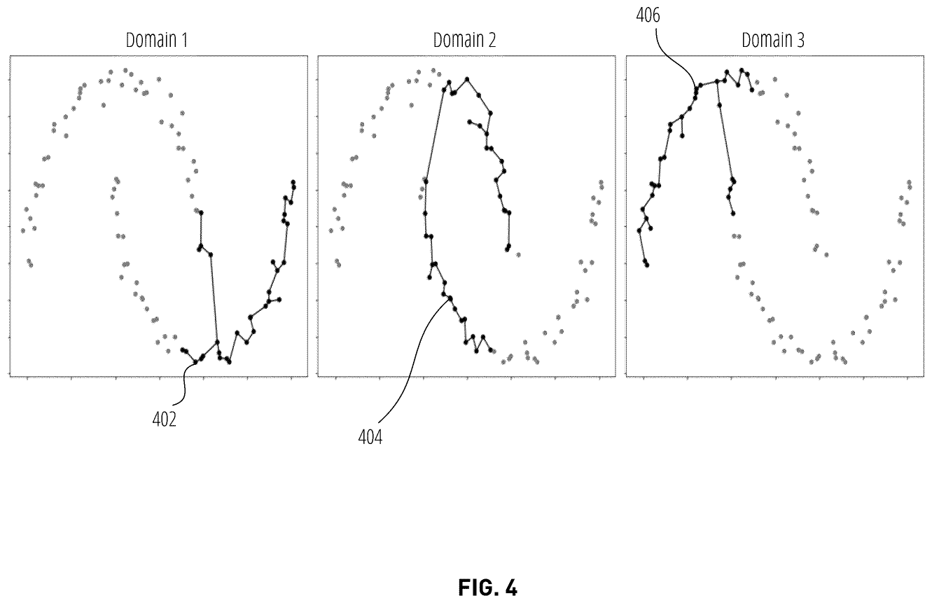

FIG. 4 illustrates minimum spanning trees for three overlapping domains, according to an example embodiment. Specifically, FIG. 4 shows minimum spanning tree 402 in an example domain 1, minimum spanning tree 404 in an example domain 2, and minimum spanning tree 406 in an example domain 3.

After determining the minimum spanning trees for each overlapping domains, the lengths of the edges of each of these trees can be considered as a sample of real numbers. The sample may include outliers. In some embodiments, the Chauvenet criterion can be applied to the sample to find the outliers. The Chauvenet criterion defines the statistically acceptable spread around the mean for a given sample of values. The real numbers (lengths of edges) that deviate significantly from the mean can be considered outliers. According to the Chauvenet criterion, all real numbers in the sample that fall within a range around the mean that corresponds to a probability of 1−1/(2N) should be retained, where N is the number of real numbers in the sample. In other words, real numbers (lengths of edges) can be considered outliers if the probability of deviation of the real numbers from the mean is less than 1/(2N).

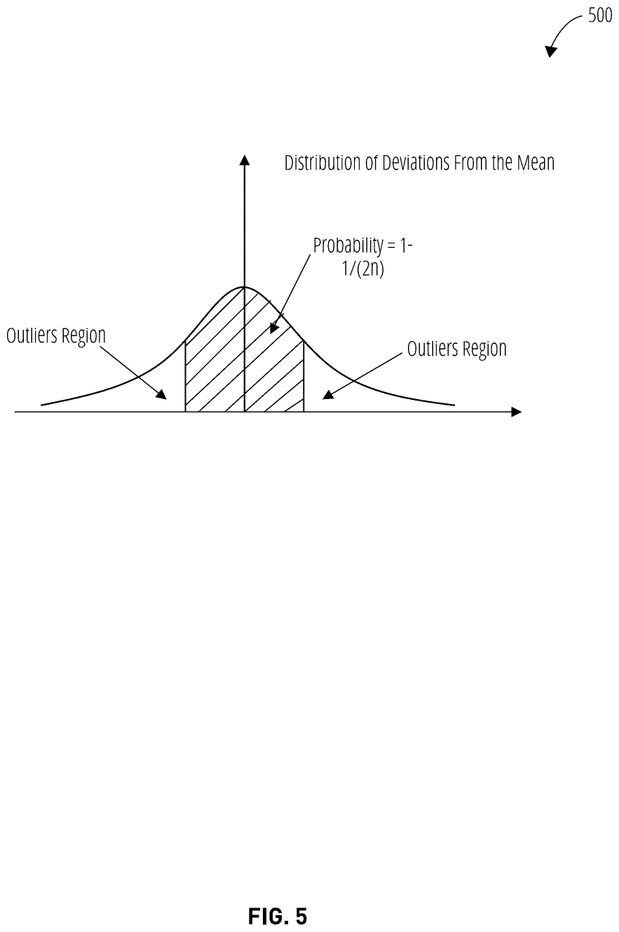

FIG. 5 illustrates an example plot 500 of the distribution of the lengths of the edges, probability of deviation of lengths of edges from the mean, and regions for outliers, according to an example embodiment.

It can be assumed that the sample data (length of the edges) comes from some distribution, for example, normal, γ-distribution, Weibull distribution, logistic, or other. Based on the sample data, the distribution parameters can be determined using the Maximum methods Likelihood Estimation (MLE) or Method of Moments (MM). Then, the Cumulative Distribution Function (CDF) for each sample point can be calculated. The points for which the CDF is less than the criterion 1/(2N) are marked as outliers. The outliers can be excluded from the sample. The length of the maximum edge from the sample that remained can be assigned as the cutoff threshold.

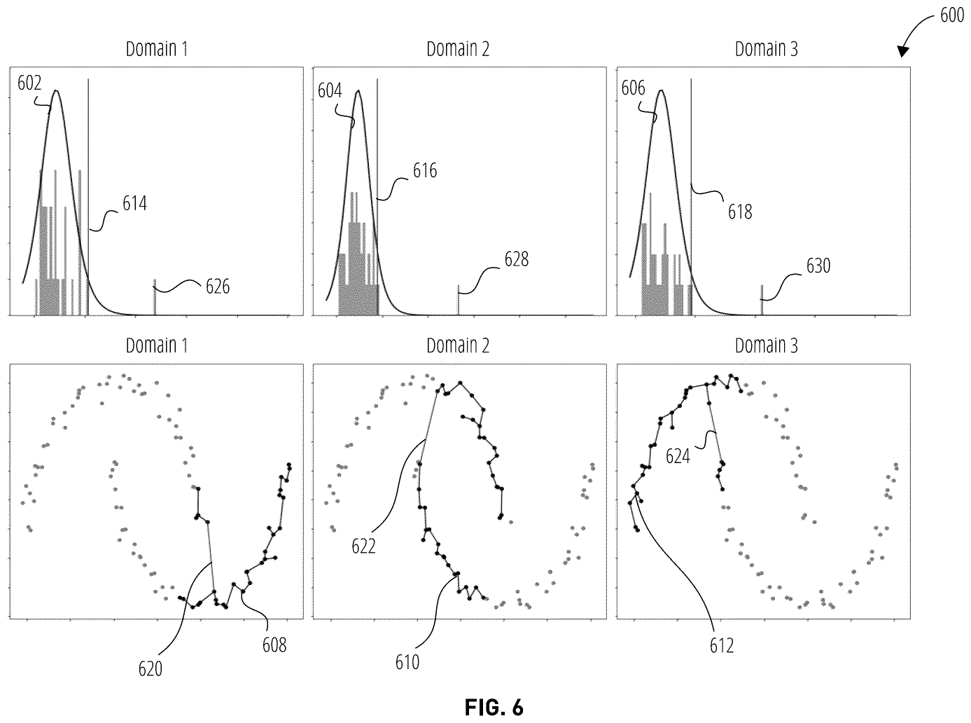

FIG. 6 is a schematic diagram 600 illustrating minimum spanning trees for three overlapping domains and the corresponding plots of distributions of the lengths of the edges in the minimum spanning trees, according to an example embodiment. Specifically, FIG. 6 shows, for domain 1, a minimum spanning tree 608, a distribution 602 for minimum spanning tree 608, a cutoff threshold 614, an outlier 626, and an edge 620 having the length corresponding to outlier 626. FIG. 6 depicts, for domain 2, a minimum spanning tree 610, a distribution 604 for minimum spanning tree 610, a cutoff threshold 616, an outlier 628, and an edge 622 having the length corresponding to outlier 628. FIG. 6 also shows, for domain 3, a minimum spanning tree 612, a distribution 606 for minimum spanning tree 612, a cutoff threshold 618, an outlier 630, and an edge 624 having the length corresponding to outlier 630.

When determining the cutoff threshold for an overlapping domain, the overlapping domain adjacent to this overlapping domain can be taken into account. For example, to obtain smoothly varying cutoff thresholds over the overlapping domains, the lengths of the edges of the minimum spanning trees of adjacent overlapping domains can be added to the sample before applying the Chauvenet criterion. The Chauvenet criterion can also be strengthened by taking values less than 1/(2N). In this case, fewer outliers can be found. Accordingly, the cutoff threshold increases, which can lead to more edges in the metric graph graph and, possibly, the metric graph will have fewer connected components.

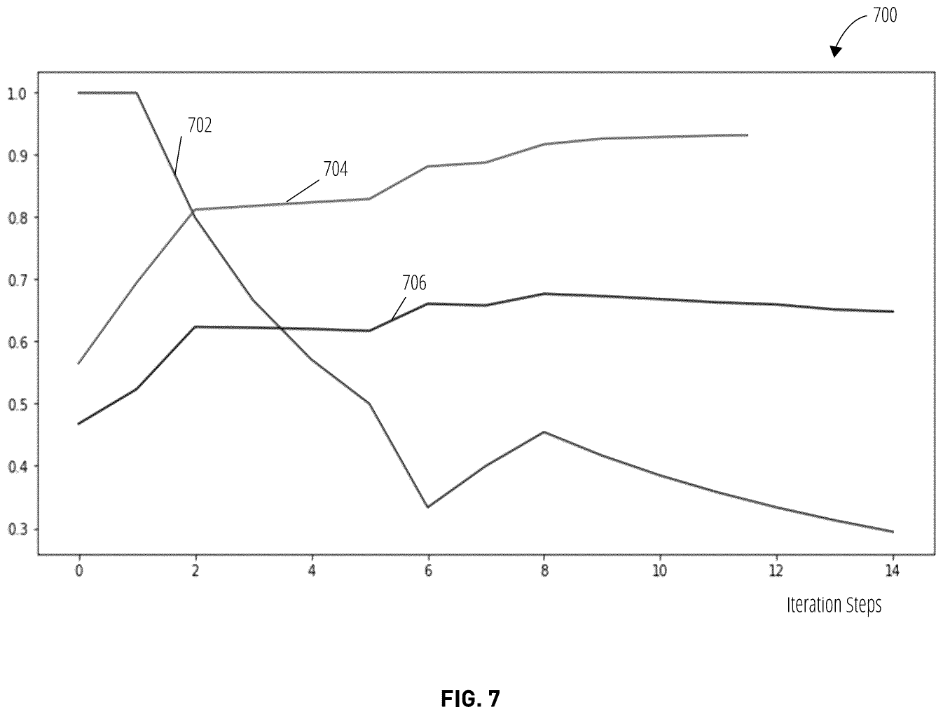

FIG. 7 is schematic diagram 700 illustrating plots of metrics corresponding to iterations of an example method for determining communities in a metric graph, according to an example embodiment. As described above in connection with FIG. 2, a clustered graph of a metric graph can be obtained as a graph of communities (groups of nodes) within the metric graph. One or more methods can be used to identify these communities, such as—but not limited to—the Girvan-Newman method, the percolation method, and the walktrap method.

The Girvan-Newman method is a community detection algorithm that identifies communities by progressively removing edges from the network. It does this by calculating the edge betweenness centrality for all edges, then removing the edge with the highest betweenness. As high-betweenness edges typically connect different communities, their removal gradually separates the network into distinct clusters or communities.

The percolation method (also known as the clique percolation method) detects communities by searching for overlapping groups of nodes that form cliques (fully connected subgraphs). Communities are identified as sets of cliques that share common nodes. This method allows for the detection of overlapping communities, acknowledging that nodes may belong to more than one community.

The walktrap method is a community detection algorithm based on random walks assuming that short random walks tend to remain within the same community. By simulating random walks of a fixed length and computing distances between nodes based on the probability of reaching one node from another, this algorithm uses a hierarchical clustering approach to group nodes into communities. This method effectively captures the local structure of the network.

The Girvan-Newman method, the percolation method, and the walktrap method have parameters that affect the number of communities. In order to determine the optimal values of these parameters, one can use the elbow-knee method on metrics such as the outcome score, performance, and modularity of a partition of the metric graph into communities.

The outcome score is a statistical measure that characterizes outcomes, for example, the number of statistically significant differences between a community's outcomes and those of the rest of the data, normalized by the number of communities.

The performance of a partition is defined as the sum of the number of intra-community edges and inter-community non-edges divided by the total number of potential edges.

Modularity compares the density of edges within communities to the density of edges expected in a random graph with the same degree distribution. Higher values for these metrics indicate a stronger community structure.

In example of FIG. 7, outcome score 702, performance 704, and modularity 706 are determined during iterations of the Girvan-Newman method for an example metric graph. By applying the elbow-knee method, either iterative step 2 or 6 can be selected. The values of the metrics at these iterative steps can be selected as optimal because it ensures a balance between improving the metric and maintaining a manageable number of communities, avoiding unnecessary complexity.

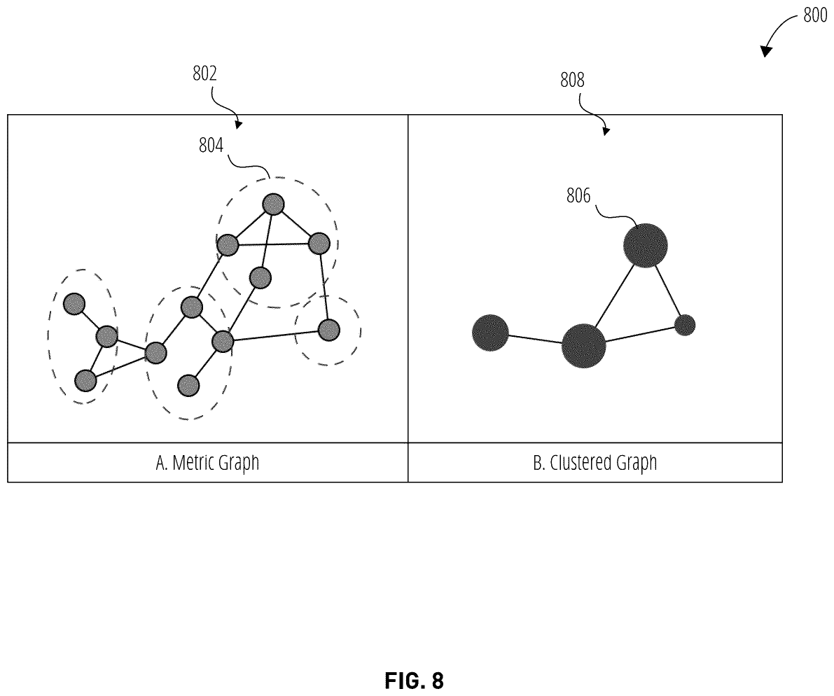

FIG. 8 is a schematic diagram 800 showing an example metric graph 802 and corresponding clustered graph 808, according to an example embodiment. In metric graph 802, each unique node corresponds to a vector of outcome of a subject trial, which a data point in a multidimensional space. Nodes that are close according to a distance or similarity measure are connected by edges. In a clustered graph 808—, several similar nodes of the metric graph (for example nodes 804) are merged into one node (for example, node 806). Nodes of the metric graph are considered part of a node in the clustered graph. Two nodes, v₁ and v₂, of the clustered graph are connected by an edge if there exist nodes in the metric graph 802 assigned to nodes v₁ and v₂ of the clustered graph 808, respectively, that are connected by an edge.

In some embodiments, clustered graphs can be constructed based on a resolution parameter (also referred to as a “scale parameter”) α (0<α≤1). The number of nodes in the clustered graph is approximately equal to αN, where N is the number of nodes of the metric graph. When α=1 the clustered graph coincides with the corresponding metric graph. The resolution parameter can be obtained from a user via a user interface. A clustered graph constructed using the resolution parameter α can be referred to as a scaled clustered graph.

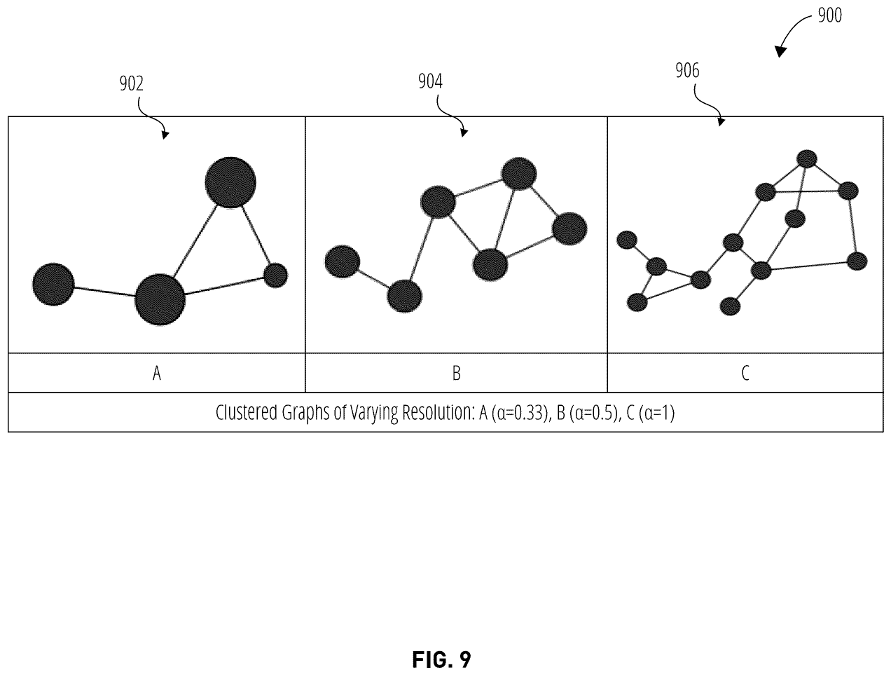

FIG. 9 is a schematic diagram 900 showing scaled clustered graphs 902, 904, and 906 corresponding the metric graph 802 of FIG. 8, according to an example embodiment. Scaled clustered graph 902 corresponds to resolution parameter α=0.33, scaled clustered graph 904 corresponds to resolution parameter α=0.5, and scaled clustered graph 906 corresponds to resolution parameter α=1. Nodes of scaled clustered graphs 902, 904, and 906 correspond to some groups of metric graph 802. An edge in scaled clustered graphs 902, 904, and 906 is drawn if the nodes of metric graph 802 in different groups are connected by edges, and the more edges of a metric graph between groups, the greater the weight of an edge of the scaled clustered graph. Scaled clustered graph 906 constructed with resolution parameter α=1 coincides with metric graph 802.

A clustered graph can be built taking into account the covering structure of the values of the projections of nodes onto overlapping domains (described in connection with FIG. 3) using the following steps.
1. Select a resolution parameter α∈(0, 1]. As shown in FIG. 9, larger values of the resolution parameter α lead to more nodes in the clustered graph, while smaller values of a result in fewer nodes. For α=1, the clustered graph coincides with the metric graph.
2. In each domain of the covering of the projection values, remove from the minimum spanning tree (described in FIG. 3-4) as many edges as necessary—starting with the longest edge (i.e., the one with the largest weight) and proceeding in decreasing order—until the number of connected components in the resulting collection of trees equals to round(α*len(nodes_in_interval)).
3. Because the domains overlap, some of the components defined above may intersect. Therefore, for any data point in the intersection, the average distance to the nearest neighbors in the group of nodes corresponding to each pair of overlapping components is calculated, and the point is assigned to the component with the smallest average distance.
4. Two groups are considered connected by an edge if there exists an edge in the metric graph that connects at least one node from one group to at least one node from the other group.

A clustered graph can also be constructed without considering the covering structure of node projection values on overlapping domains, instead relying on communities detected in a metric graph.

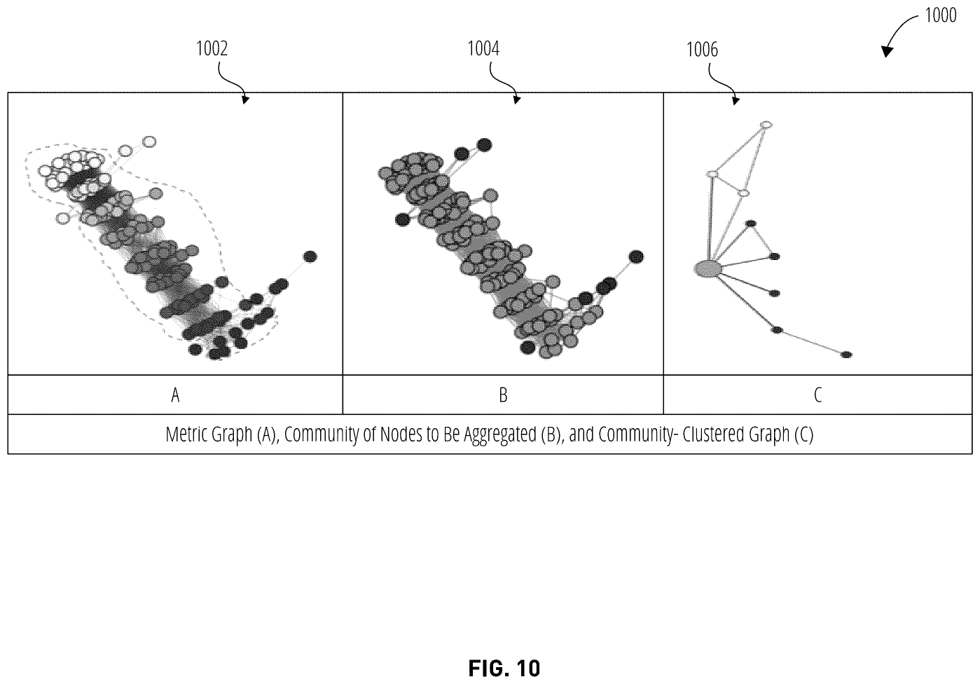

FIG. 10 is a schematic diagram 1000 showing an example metric graph 1002, communities 1004, and corresponding community-clustered graph 1006, according to an example embodiment. Each node in the community-clustered graph 1006 corresponds to a community of communities 1004 of nodes in the metric graph 1002. In drawing A, different communities of nodes in the metric graph 1002 are depicted using different colors, while in drawing B, they are shown in the same color.

Communities in the metric graph can be identified by methods described in FIG. 7, such as Girvan-Newman method, the percolation method, and the walktrap method. Some nodes may not belong to any community, as observed with the percolation method. Using this community structure, a version of a clustered graph—referred to as the community-clustered graph—can be constructed. Each community is then replaced by a single aggregated node, using the same algorithm as that used in constructing the scaled clustered graph. The community-based clustered graph is not governed by a resolution parameter α, but rather by the set of communities that are aggregated into nodes. Community-clustered graphs are used for simplified visualization of the connections between communities or between a community and a non-community node in the metric graph. In this approach, a community can be treated as a single node.

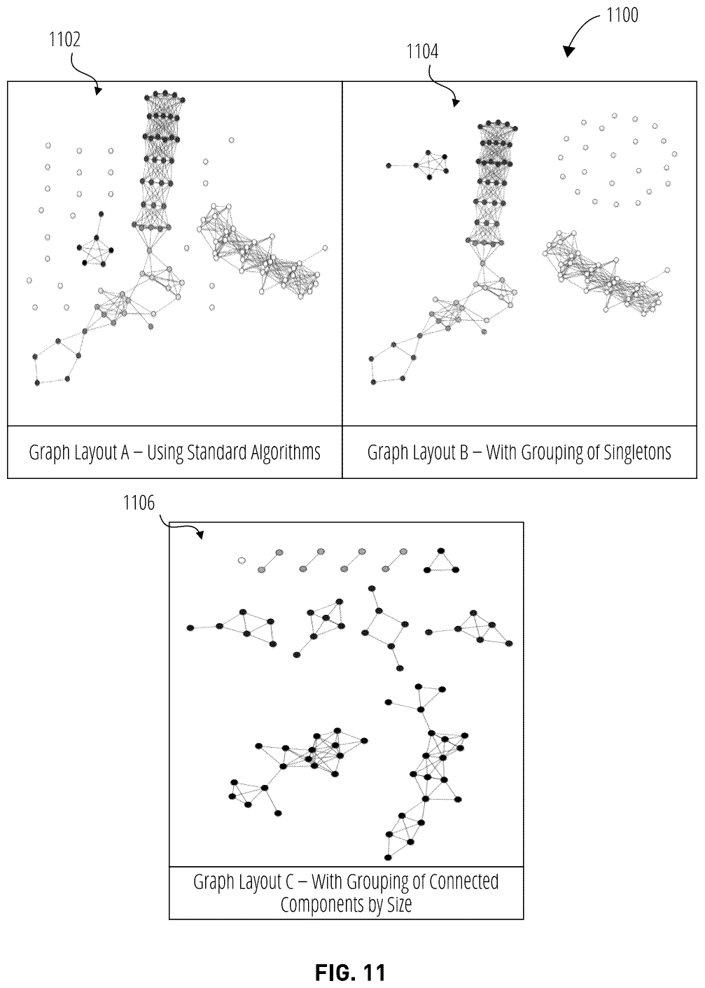

FIG. 11 is a schematic diagram 1100 showing different layouts of metric graphs, according to some example embodiments. A layout of a metric graph can be referred as a planar or spatial arrangement that assigns coordinates to each node in the metric graphs so that the distances between nodes visually reflect the underlying metric relationships derived from the data. The layouts can be computed by different algorithms that determine the coordinates for each node in two-dimensional or three-dimensional space. In some embodiments, layouts of arbitrary graphs are constructed using force-directed placement (FDP) algorithms. These algorithms interpret the graph as a physical system in which nodes are modeled either as particles subject to both attractive and repulsive forces or as elastic rings interconnected by spring-like forces along the edges. In these algorithms, computing the layout is equivalent to finding the equilibrium state of the system. For connected graphs, the equilibrium configuration can be computed directly. For graphs comprising multiple connected components, the layout of each component is determined independently, and the resulting components can be subsequently consolidated into a single comprehensive layout. One exemplary consolidation technique is the polyomino algorithm, which arranges the connected components into checkered figures (such as rectangles or more complex polyominoes) and then positions these figures as closely as possible to one another.

In some embodiments of the present disclosure embodiment, the layout computation algorithm incorporates distinct features arising from the metric graph's representation as a data map. The algorithm for determining the packing of a metric graph can be configurable, enabling various strategies for arranging its connected components based on specific application requirements, where connected components are subsets of nodes in which any pair of nodes is directly or indirectly connected by edges, and which are disconnected from other nodes in the graph. For example:
1. Individual nodes (or singletons) can be aggregated within a designated area of the canvas into a predetermined shape, such as a circle or square, to emphasize the most significant connectivity components.
2. Connected components may be ordered by size, with components of similar sizes grouped into rows to facilitate the identification of comparably sized clusters.
3. Alternatively, connected components of the metric graph can be organized according to the average value of a particular feature distributed across the nodes.

FIG. 11 illustrates layout 1102, where the singletons are arranged without grouping, and layout 1104, where the singletons are grouped, and layout 1106, where the connected components are grouped based on the size of the connected components.

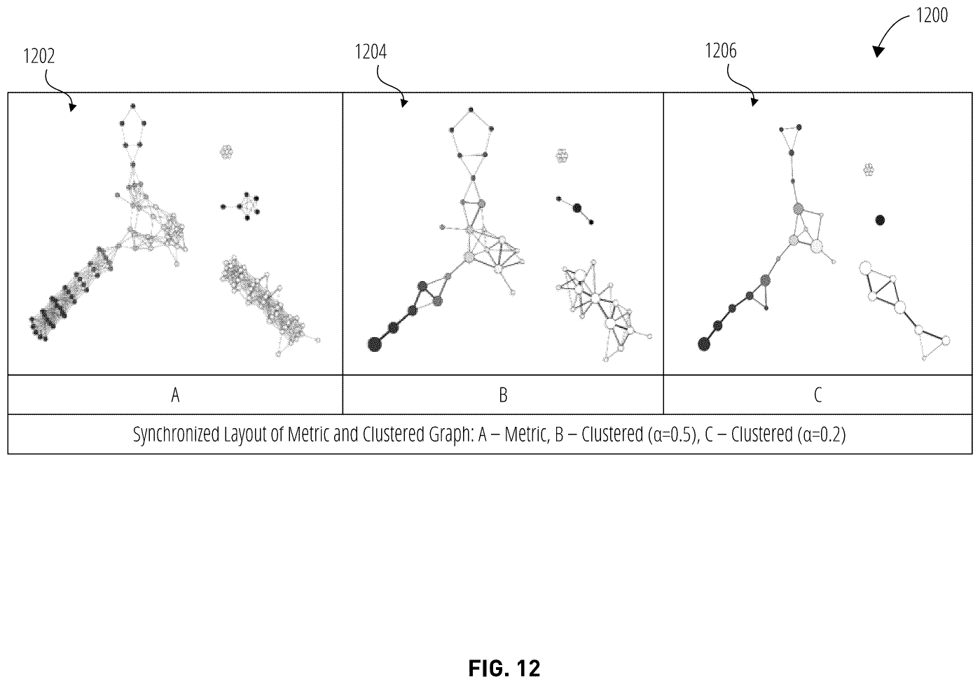

FIG. 12 is a schematic diagram 1200 illustrating a layout 1202 of a metric graph and two synchronized layouts 1204 and 1206 of clustered graphs corresponding to the metric graph, according to an example embodiment. Layout 1204 represents a clustered graph obtained from the metric graph using resolution parameter α=0.5. Layout 1206 represents a clustered graph obtained from the metric graph using resolution parameter α=0.2.

As seen in FIG. 12, the nodes of a metric graph in layout 1102 are depicted as circles of uniform radius and the edges as line segments of constant thickness. In layouts 1204 and 1206 of clustered graphs, the area of the circle representing a node v is scaled proportionally to the number of metric graph's nodes aggregated within the node v, and the thickness of an edge connecting nodes of clustered graphs is scaled in proportion to the number of interconnections between the corresponding groups of metric graph's nodes.

In embodiments where the metric graph and the clustered graph are derived from the same dataset, it is essential that their respective layouts remain synchronized, that is the planar arrangement of the nodes in both layouts is consistently aligned, such that corresponding nodes or components in both layouts maintain a consistent relative positioning or connection structure, reflecting the same underlying data or relationships.

Because the metric graph and the clustered graph are derived from the same dataset, their respective packings needed to be synchronized. To achieve this, nodes in the metric graph are linked to their corresponding nodes in the clustered graph via additional connecting edges, resulting in a unified, synchronized layout.

The method for constructing the synchronous layout comprises the following steps:
1. Generate a layout for each connected component of the metric graph.
2. Establish a preliminary arrangement for the clustered graph by positioning each node v at the centroid of the associated metric graph nodes.
3. Optimize the layout of each connected component of the clustered graph using an appropriate FDP algorithm.
4. Construct a composite graph that integrates both the nodes and edges of the metric and clustered graphs, where the metric graph nodes corresponding to a clustered graph node v are linked to node v by additional edges.
5. Pack the connected components of the composite graph using the polyomino algorithm.
6. Extract the separate metric and clustered graph packings from the composite graph layout and remove the additional edges.

The synchronous layout of a metric graph and a clustered graph can be scaled to a sequence of clustered graphs of varying detail that correspond to the same metric. The same idea of linking a metric graph to each of the clustered ones using additional edges, constructing a layout of the combined graph, and then extracting the layouts of each of the graphs is used.

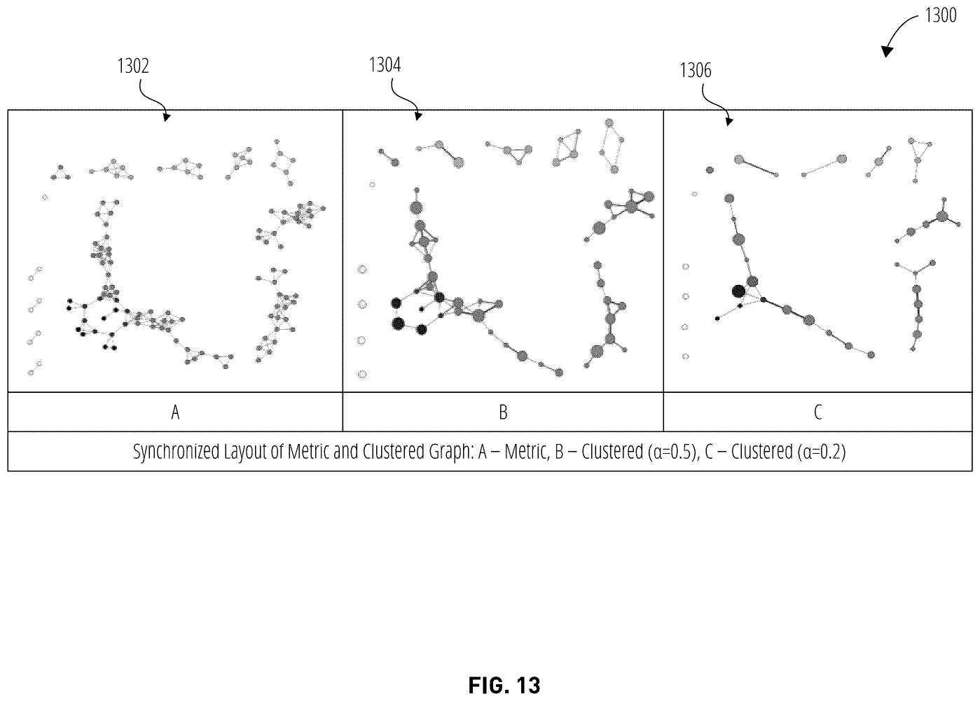

FIG. 13 is a schematic diagram 1300 illustrating a layout 1302 of a metric graph and two synchronized layouts 1304 and 1306 of clustered graphs corresponding to the metric graph, according to another example embodiment. Layout 1304 represents a clustered graph obtained from the metric graph using resolution parameter α=0.5. Layout 1306 represents a clustered graph obtained from the metric graph using resolution parameter α=0.2.

The initial positioning of graph nodes can be critical to the performance of FDP algorithms that are used to generate the layouts. An optimal initial configuration enables faster computation and higher-quality layouts, whereas a poor configuration may increase processing time and degrade layout quality. Traditionally, nodes for an arbitrary graph are randomly positioned. However, the inherent characteristics of a metric graph permit a more deliberate selection of initial positions, thereby enhancing the efficiency and effectiveness of the force-directed layout algorithm.

According to some embodiments of the present disclosure, the algorithm for constructing a metric graph layout includes, in addition to random initialization, the following methods for determining initial nodes positions:
1. Interval-Based Placement: Nodes are divided into intervals according to their projection values during the construction of the metric graph. Within each interval, the initial positions of the nodes are assigned with additional random perturbations.
2. Clustered Graph-Based Placement: Since a clustered graph contains fewer nodes than a metric graph, its layout can be computed more rapidly. The layout of the clustered graph is used to initialize the positions of the corresponding metric graph nodes. Specifically, for each node v in the clustered graph, the metric graph nodes associated with v are randomly distributed within a small-radius disk centered at the position of node v. Furthermore, if a series of clustered graphs with varying degrees of detail is available (found with different resolution parameters), the layout of a more detailed graph can be initialized by positioning its vertices near those of a coarser graph, after which the layout optimization algorithm is applied. This multilevel layout approach can be particularly effective for graphs with a large number of vertices.
3. Dimensionality Reduction-Based Placement: Dimensionality reduction algorithms—such as Principal Component Analysis, Multidimensional Scaling, t-Distributed Stochastic Neighbor Embedding, and Uniform Manifold Approximation and Projection—can be employed to compute projections of the original dataset onto a plane or into three-dimensional space. These projections can be used as the initial positions for the vertices in the layout computation.

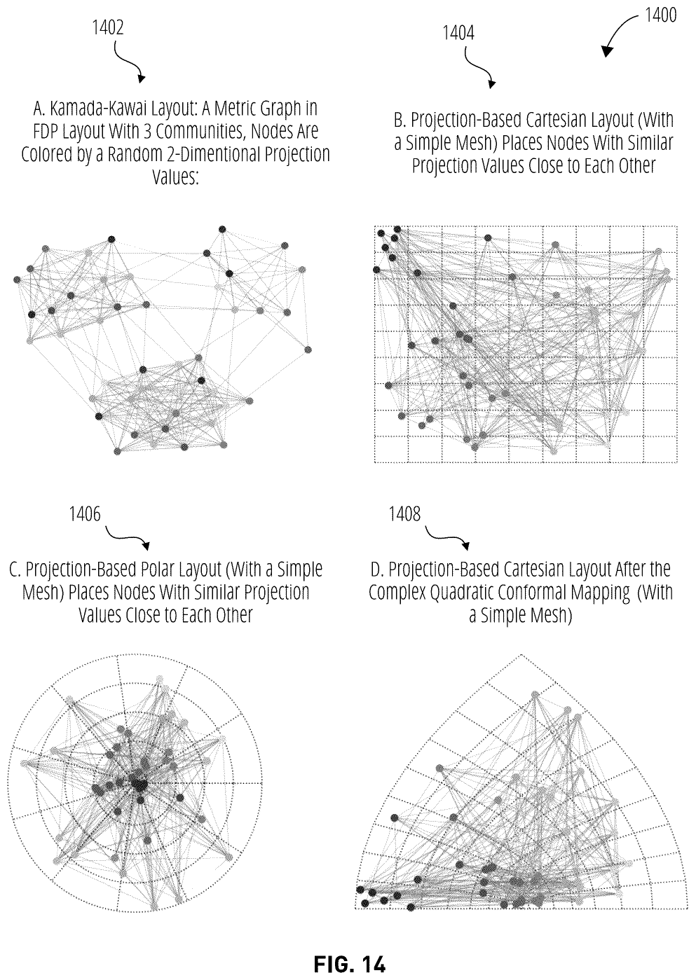

FIG. 14 is a schematic diagram 1400 showing layouts of metric graphs with applying different conformal mappings, according to some example embodiments. Conformal mappings can be referred to as mathematical functions that preserve the angles between curves while potentially altering the sizes or shapes of objects.

Graph layouts employing conformal mapping can be based on the projections of nodes (i.e., outcome vectors) to overlapping domains described in connection with FIG. 3.

In some embodiments, for one-dimensional projections, graph nodes are positioned along the x-axis based on their one-dimensional projection values, which serve as the x-coordinates. The y-coordinate can then be determined through algorithms, such as force-directed placement. Alternatively, the y-coordinate may be derived using additional projection methods, such as Multidimensional Scaling (MDS)—a dimensionality reduction technique designed to project high-dimensional data into a lower-dimensional space while preserving pairwise distances—or Principal Component Analysis (PCA) projection, which identifies the component with the greatest variance, capturing the most significant information from the original dataset.

In the embodiments involving two-dimensional projections, the x- and y-coordinates of each node correspond directly to the respective projection values. Covering overlapping domains, or levels, may be visualized as a mesh, with each cell containing nodes from the corresponding overlapping domain.

Alternatively, instead of using a Cartesian coordinate system (x, y) on the plane, polar coordinates (r, q) can be employed to represent the projection values. In such cases, the partitioning of the plane into stripes (representing one-dimensional covering) is replaced by a partitioning into rings. In general, a conformal mapping—or another suitable mapping—that transforms a point z=(x, y) into a point w=(u, v) can be applied to the graph layout.

In one embodiment, overlapping domains (or levels) are visualized as stripes superimposed on the graph layout, with the option to display or hide the projection axes based on user preference. The width of each stripe or ring is determined by factors such as the number of nodes, their diameters and placements, and the normalization of the projection data. Similarly, the grid step is defined according to the covering, the number of nodes, and their spatial arrangement. The grid visualization is configurable according to specific requirements. For example, the grid may be overlaid to illustrate overlapping intervals, partitioned into a disjoint union by segmenting the covering with overlap, or represented as a simple mesh that reflects the geometry of the selected layout and its organization according to coordinate lines corresponding to projection value levels.

FIG. 14 illustrates the following graph layouts that employ conformal mapping techniques. Layout 1402 is a Kamada-Kawai layout—representing a metric graph arranged by a force-directed placement algorithm and organized into three communities—in which nodes are colored according to randomly generated two-dimensional projection values. Layout 1404 is a projection-based Cartesian layout, with an overlaid simple mesh, that positions nodes with similar projection values in close proximity to each other. Layout 1406 is a projection-based polar layout with a simple mesh, wherein nodes having close projection values are close to each other. Layout 1408 is a projection-based Cartesian layout that utilizes a complex quadratic conformal mapping (along with a simple mesh). A complex quadratic conformal mapping can be defined as a mapping in the complex plane given by a quadratic polynomial—such as f(z)=az²+bz+c, where a, b, and c are complex numbers with a being different from zero—to preserve angles and local geometric shapes.

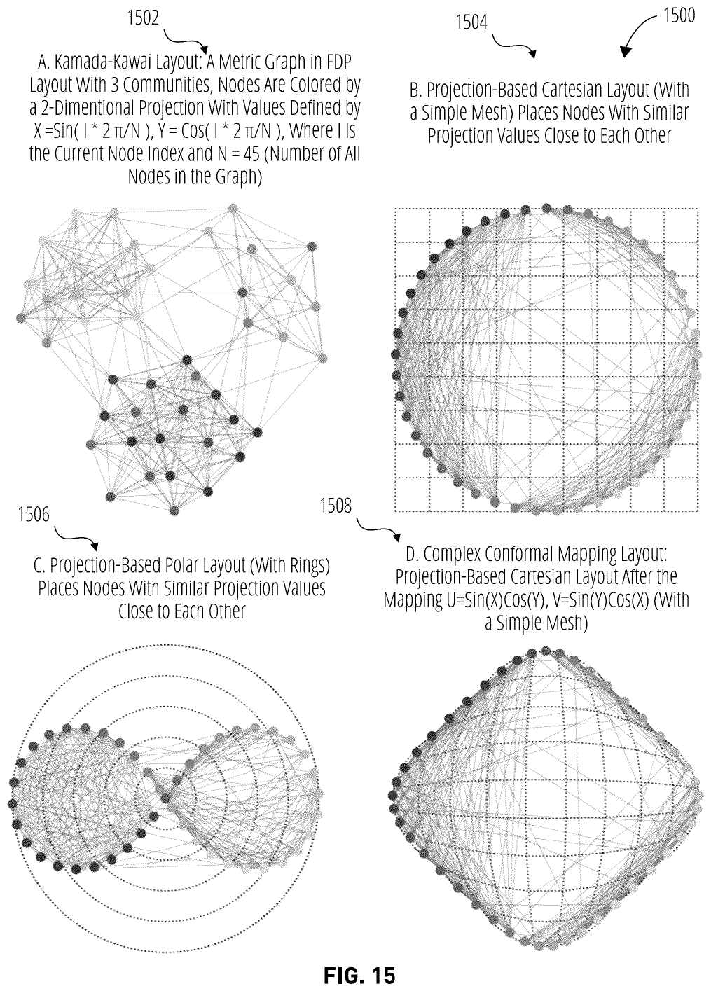

FIG. 15 is a schematic diagram 1500 showing layouts of metric graphs with applying different conformal mappings, according to another example embodiment. In FIG. 15, layout 1502 is a metric graph in FDP layout with 3 communities, nodes are colored by a 2-dimentional projection with values defined by x=sin(i·2π/n), y=cos(i·2π/n), where i is the current node index and n=45 (number of all nodes in the graph).

Layout 1504 is a projection-based Cartesian layout (with a simple mesh) that places nodes with similar projection values close to each other.

Layout 1506 is a projection-based polar layout (with rings) places nodes with similar projection values close to each other.

Layout 1508 is a projection-based Cartesian layout after the mapping u=sin(x)cos(y), v=sin(y)cos(x) (with a simple mesh).

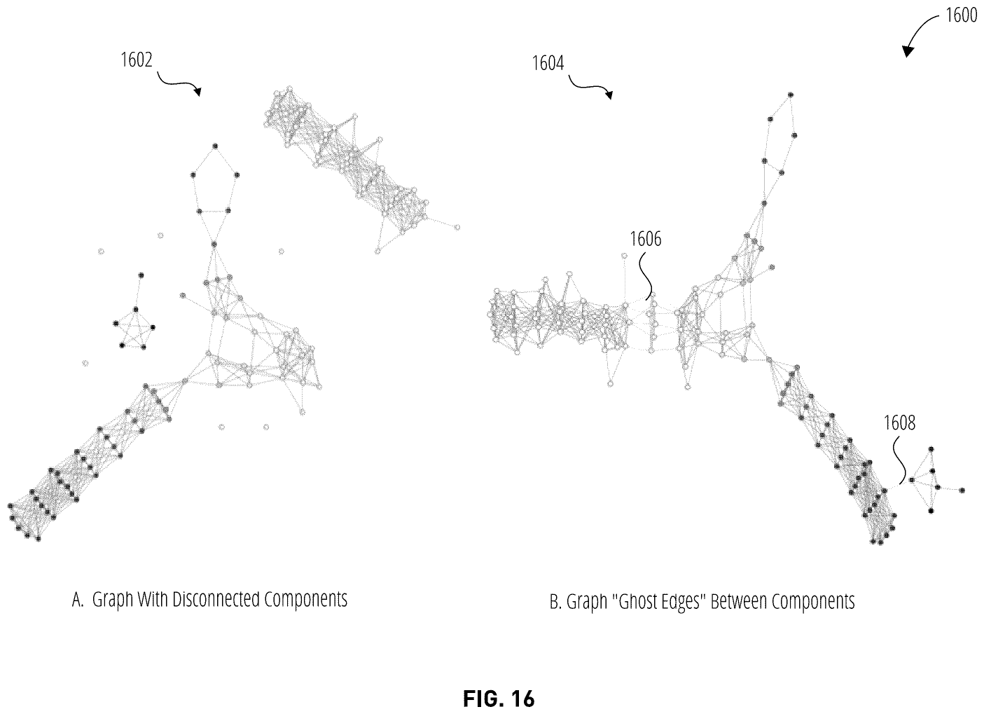

FIG. 16 is a schematic diagram 1600 depicting two layouts of a metric graph, according to an example embodiment. In FIG. 16, layout 1602 represents a metric graph, where connected components are separate from each other. Layout 1604 represents the same metric graph, where the originally disconnected components are connected with “ghost edges” 1606 and 1608.

The “ghost edges” may indicate which of the connected components correspond to which parts of the original metric graph. In one embodiment, connectivity is achieved by adding ghost edges. To add the “ghost edges” to graph M, an auxiliary graph G is constructed, wherein each node vᵢ corresponds to a connected component Vᵢ of the graph M, and each edge eᵢⱼ connecting nodes vᵢ and vⱼ corresponds to one or more edges [aᵢ, aⱼ] in M that connect the connected components containing aᵢ and aⱼ, respectively. Each edge eᵢⱼ in G is assigned the following attributes:
‘d’: the distance between aᵢ and aⱼ;
‘inds’: a list of indices of the vertices in M that are to be connected by ghost edges; and
‘levels’: a list of projection intervals in which the ghost edges reside.

Initially, any disconnectedness occurring within a single overlapping domain (a projection interval) is eliminated by adding edges to G inductively, as follows:
1. For each interval, iterate over the connected components within that interval:
i. If only one connected component exists in the interval, no further action is taken;
ii. Otherwise, identify a pair of nodes aᵢ and aⱼ located in different connected components that exhibit the minimum distance between them (or identify multiple pairs if the minimum distance is identical). An edge eᵢⱼ is then added or updated in the connectivity component graph with the corresponding attribute values.
2. After constructing the edges of G, ghost edges are added to the original graph M utilizing the information specified in ‘inds.’

In certain embodiments, if one of the intervals in the covering of projection values is empty, the graph may remain disconnected. In such cases, adjacent nonempty intervals are identified. Within these adjacent intervals, any gap is addressed by determining the closest points and connecting them with an edge with following steps.
1. An auxiliary graph of adjacent intervals is constructed, in which the nodes represent the intervals, and an edge is drawn between intervals that are adjacent (i.e., have a nonempty intersection).
2. Pairs of nearest intervals belonging to different connected components are then identified using the graph of neighboring intervals. This is accomplished by iterating over pairs of connected components. For the component containing fewer intervals, each interval is examined to find the closest interval in the other connected component using the neighboring intervals graph. For each identified pair of intervals—one from each connected component—a pair of points with the minimum distance between them (or multiple pairs if the distance is identical) is determined, and an edge is added or updated in the connectivity component graph accordingly.
3. After updating the graph of connectivity components, additional edges are incorporated into the original graph. The layout is then computed for the resulting graph (i.e., the original graph with the ghost edges), and the ghost edges are rendered in a different color or with a different line style.

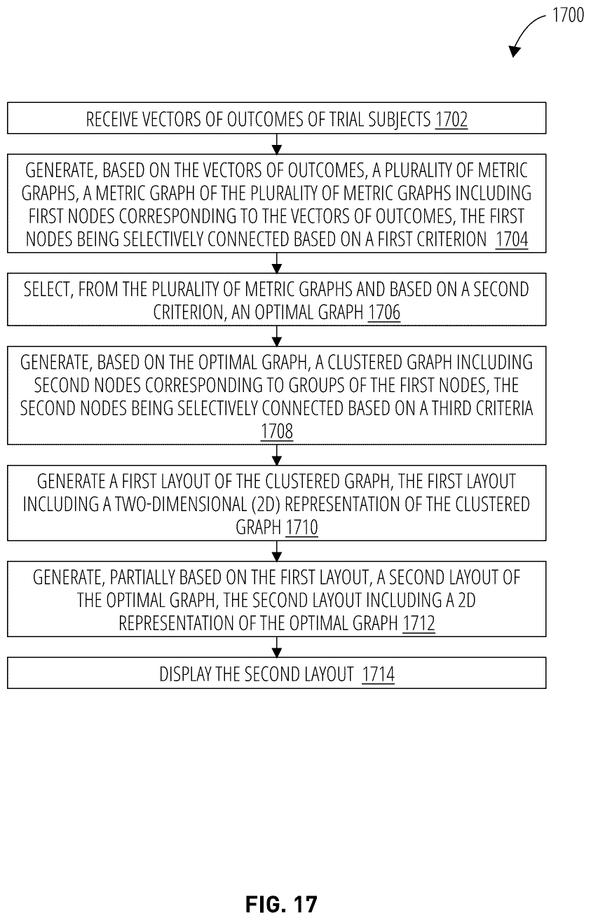

FIG. 17 is a process flow diagram showing a method 1700 for multiscale graph-based analysis and visualization of clinical data, according to an example embodiment. The method 1700 may be performed by processing logic that comprises hardware (e.g., decision-making logic, dedicated logic, programmable logic, ASIC, and microcode), software (such as software run on a general-purpose computer system or a dedicated machine), or a combination of both. The method 1700 may have additional operations not shown herein, but which can be evident to those skilled in the art from the present disclosure. The method 1700 may also have fewer operations than outlined below and shown in FIG. 17.

In block 1702, method 1700 may include receiving vectors of outcomes of trial subjects. In block 1704, method 1700 may include generating, based on the vectors of outcomes, a plurality of metric graphs, a metric graph of the plurality of metric graphs including first nodes corresponding to the vectors of outcomes, the first nodes being selectively connected based on a first criterion.

In block 1706, method 1700 may include selecting, from the plurality of metric graphs and based on a second criterion, an optimal graph. In block 1708, method 1700 may include generating, based on the optimal graph, a clustered graph. The clustered graph may include second nodes corresponding to groups of the first nodes, the second nodes being selectively connected based on a third criteria.

In block 1710, method 1700 may include generating a first layout of the clustered graph, the first layout including a two-dimensional (2D) representation of the clustered graph. Method 1700 may include, prior to the generating the clustered graph, the following: receiving a resolution parameter and determining the groups of the first nodes based on the resolution parameter. The resolution parameter can be received from a user via a user interface. The determination of the groups of the first nodes can include the following: projecting the first nodes onto a set of domains and determining a tree including the first nodes having projections in a domain of the set of domains, such that the tree has a minimum sum of distances between the first nodes connected in the tree. The determination of the groups of the first nodes can then include determining, based on the tree and the resolution parameter, subtrees of the tree and forming the groups based on the subtrees. A number of subtrees is selected based on a product of the resolution parameter and a number of the first nodes having projections in the domain of the set of domains.

In block 1712, method 1700 may include generating, partially based on the first layout, a second layout of the optimal graph, the second layout including a 2D representation of the optimal graph. The second layout can be determined by an iteration procedure starting with an approximate layout. The approximate layout can be determined based on the first layout of the clustered graph.

In block 1714, method 1700 may include displaying the second layout. Method 1700 may further include displaying the first layout of the clustered graph synchronously with the second layout of the optimal graph.

Method 1700 may also include determining that the optimal graph includes a first subgraph and a second subgraph, where nodes of the first subgraph disconnected from further nodes of the second subgraph. Method 1700 may include adding a connection between a node of the first subgraph and a further node of the second subgraph and displaying the connection using one of the following: a line and a collection of lines. At least one characteristic of the line can differ from characteristics of a further line, where the further line is used to display one of the following: a connection between the nodes of the first subgraph and a connection between the nodes of the second subgraph.

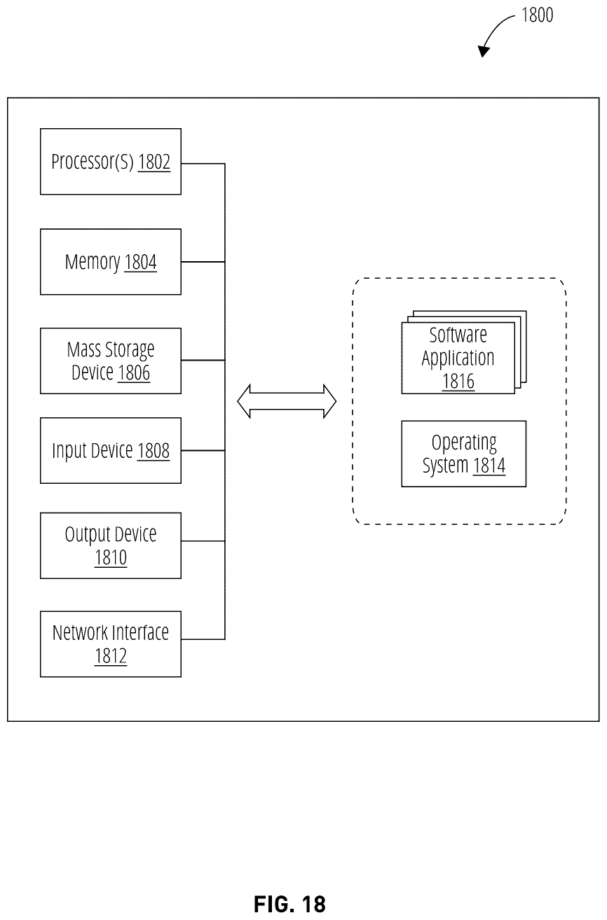

FIG. 18 is a high-level block diagram illustrating an example computer system 1800, within which a set of instructions for causing the machine to perform any one or more of the methodologies discussed herein can be executed. The computer system 1800 may include, refer to, or be an integral part of, one or more of a variety of types of devices, such as a general-purpose computer, a desktop computer, a laptop computer, a tablet computer, a netbook, a mobile phone, a smartphone, a personal digital computer, a smart television device, and a server, among others. In some embodiments, the computer system 1800 is an example of a computing system 110, clinical datasets sources 105, user computing device(s) 125 shown in FIG. 1. Notably, FIG. 18 illustrates just one example of the computer system 1800 and, in some embodiments, the computer system 1800 may have fewer elements/modules than shown in FIG. 18 or more elements/modules than shown in FIG. 18.

The computer system 1800 may include one or more processor(s) 1802, a memory 1804, one or more mass storage devices 1806, one or more input devices 1808, one or more output devices 1810, and a network interface 1812. The processor(s) 1802 are, in some examples, configured to implement functionality and/or process instructions for execution within the computer system 1800. For example, the processor(s) 1802 may process instructions stored in the memory 1804 and/or instructions stored on the mass storage devices 1806. Such instructions may include components of an operating system 1814 or software applications 1816. The computer system 1800 may also include one or more additional components not shown in FIG. 18, such as a body, a power supply, a power supply, a global positioning system (GPS) receiver, and so forth.

The memory 1804, according to one example, is configured to store information within the computer system 1800 during operation. The memory 1804, in some example embodiments, may refer to a non-transitory computer-readable storage medium or a computer-readable storage device. In some examples, the memory 1804 is a temporary memory, meaning that a primary purpose of the memory 1804 may not be long-term storage. The memory 1804 may also refer to a volatile memory, meaning that the memory 1804 does not maintain stored contents when the memory 1804 is not receiving power. Examples of volatile memories include random access memories (RAM), dynamic random access memories (DRAM), static random access memories (SRAM), and other forms of volatile memories known in the art. In some examples, the memory 1804 is used to store program instructions for execution by the processor(s) 1802. The memory 1804, in one example, is used by software (e.g., the operating system 1814 or the software applications 1816). Generally, the software applications 1816 refer to software Applications suitable for implementing at least some operations of the methods for multiscale graph-based analysis and visualization of clinical data as described herein.

The mass storage devices 1806 may include one or more transitory or non-transitory computer-readable storage media and/or computer-readable storage devices. In some embodiments, the mass storage devices 1806 may be configured to store greater amounts of information than the memory 1804. The mass storage devices 1806 may further be configured for long-term storage of information. In some examples, the mass storage devices 1806 include non-volatile storage elements. Examples of such non-volatile storage elements include magnetic hard discs, optical discs, solid-state discs, flash memories, forms of electrically programmable memories (EPROM) or electrically erasable and programmable memories, and other forms of non-volatile memories known in the art.

The input devices 1808, in some examples, may be configured to receive input from a user through tactile, audio, video, or biometric channels. Examples of the input devices 1808 may include a keyboard, a keypad, a mouse, a trackball, a touchscreen, a touchpad, a microphone, one or more video cameras, image sensors, fingerprint sensors, or any other device capable of detecting an input from a user or other source, and relaying the input to the computer system 1800, or components thereof.

The output devices 1810, in some examples, may be configured to provide output to a user through visual or auditory channels. The output devices 1810 may include a video graphics adapter card, a liquid crystal display (LCD) monitor, a light emitting diode (LED) monitor, an organic LED monitor, a sound card, a speaker, a lighting device, a LED, a projector, or any other device capable of generating output that may be intelligible to a user. The output devices 1810 may also include a touchscreen, a presence-sensitive display, or other input/output capable displays known in the art.

The network interface 1812 of the computer system 1800, in some example embodiments, can be utilized to communicate with external devices via one or more data networks such as one or more wired, wireless, or optical networks including, for example, the Internet, intranet, LAN, WAN, cellular phone networks, Bluetooth radio, and an IEEE 902.11-based radio frequency network, Wi-Fi Networks®, among others. The network interface 1812 may be a network interface card, such as an Ethernet card, an optical transceiver, a radio frequency transceiver, or any other type of device that can send and receive information.

The operating system 1814 may control one or more functionalities of the computer system 1800 and/or components thereof. For example, the operating system 1814 may interact with the software applications 1816 and may facilitate one or more interactions between the software applications 1816 and components of the computer system 1800. As shown in FIG. 18, the operating system 1814 may interact with or be otherwise coupled to the software applications 1816 and components thereof. In some embodiments, the software applications 1816 may be included in the operating system 1814. In these and other examples, virtual modules, firmware, or software may be part of the software applications 1816.

Thus, systems and methods for multiscale graph-based analysis and visualization of clinical data have been described. Although embodiments have been described with reference to specific example embodiments, it will be evident that various modifications and changes can be made to these example embodiments without departing from the broader spirit and scope of the present application. Accordingly, the specification and drawings arc to be regarded in an illustrative rather than a restrictive sense.

What is claimed is:

1. A method for analysis and visualization of clinical data, the method comprising:
receiving vectors of outcomes of trial subjects;
generating, based on the vectors of outcomes, a plurality of metric graphs, a metric graph of the plurality of metric graphs including first nodes corresponding to the vectors of outcomes, the first nodes being selectively connected based on a first criterion;
selecting, from the plurality of metric graphs and based on a second criterion, an optimal graph;
generating, based on the optimal graph, a clustered graph including second nodes corresponding to groups of the first nodes, the second nodes being selectively connected based on a third criteria;
generating a first layout of the clustered graph, the first layout including a two-dimensional (2D) representation of the clustered graph;
generating, partially based on the first layout, a second layout of the optimal graph, the second layout including a 2D representation of the optimal graph; and
displaying the second layout.

2. The method of claim 1, further comprising, prior to the generating the clustered graph,
receiving a resolution parameter; and
determining the groups of the first nodes based on the resolution parameter.

3. The method of claim 2, wherein the resolution parameter is received from a user via a user interface.

4. The method of claim 2, wherein the determining the groups includes:
projecting the first nodes onto a set of domains;
determining a tree including the first nodes having projections in a domain of the set of domains, the tree having a minimum sum of distances between the first nodes connected in the tree;
determining, based on the tree and the resolution parameter, subtrees of the tree; and
forming the groups based on the subtrees.

5. The method of claim 4, wherein a number of subtrees is selected based on a product of the resolution parameter and a number of the first nodes having projections in the domain of the set of domains.

6. The method of claim 1, wherein the second layout is determined by an iteration procedure starting with an approximate layout.

7. The method of claim 6, wherein the approximate layout is determined based on the first layout of the clustered graph.

8. The method of claim 1, further comprising displaying the first layout of the clustered graph synchronously with the second layout of the optimal graph.

9. The method of claim 1, further comprising:
determining that the optimal graph includes a first subgraph and a second subgraph, wherein nodes of the first subgraph disconnected from further nodes of the second subgraph;
adding a connection between a node of the first subgraph and a further node of the second subgraph; and
displaying the connection using one of the following: a line and a collection of lines.

10. The method of claim 9, wherein at least one characteristic of the line differs from characteristics of a further line, the further line being used to display one of the following: a connection between the nodes of the first subgraph and a connection between the nodes of the second subgraph.

11. A computing device comprising:
a processor; and
a memory storing instructions that, when executed by the processor, configure the computing device to:
receive vectors of outcomes of trial subjects;
generate, based on the vectors of outcomes, a plurality of metric graphs, a metric graph of the plurality of metric graphs including first nodes corresponding to the vectors of outcomes, the first nodes being selectively connected based on a first criterion;
select, from the plurality of metric graphs and based on a second criterion, an optimal graph;
generate, based on the optimal graph, a clustered graph including second nodes corresponding to groups of the first nodes, the second nodes being selectively connected based on a third criteria;
generate a first layout of the clustered graph, the first layout including a two-dimensional (2D) representation of the clustered graph;
generate, partially based on the first layout, a second layout of the optimal graph, the second layout including a 2D representation of the optimal graph; and
display the second layout.

12. The computing device of claim 11, wherein the instructions further configure the computing device to, prior to the generating the clustered graph,
receive a resolution parameter; and
determine the groups of the first nodes based on the resolution parameter.

13. The computing device of claim 12, wherein the resolution parameter is received from a user via a user interface.

14. The computing device of claim 12, wherein the determining the groups includes:
project the first nodes onto a set of domains;
determine a tree including the first nodes having projections in a domain of the set of domains, the tree having a minimum sum of distances between the first nodes connected in the tree;
determine, based on the tree and the resolution parameter, subtrees of the tree; and
form the groups based on the subtrees.

15. The computing device of claim 14, wherein a number of subtrees is selected based on a product of the resolution parameter and a number of the first nodes having projections in the domain of the set of domains.

16. The computing device of claim 11, wherein the second layout is determined by an iteration procedure start with an approximate layout.

17. The computing device of claim 16, wherein the approximate layout is determined based on the first layout of the clustered graph.

18. The computing device of claim 11, wherein the instructions further configure the computing device to display the first layout of the clustered graph synchronously with the second layout of the optimal graph.

19. The computing device of claim 11, wherein the instructions further configure the device to:
determine that the optimal graph includes a first subgraph and a second subgraph, wherein nodes of the first subgraph disconnected from further nodes of the second subgraph;
add a connection between a node of the first subgraph and a further node of the second subgraph; and
display the connection using one of the following: a line and a collection of lines.

20. A non-transitory computer-readable storage medium, the computer-readable storage medium including instructions that, when executed by a computing device, cause the computing device to:
receive vectors of outcomes of trial subjects;
generate, based on the vectors of outcomes, a plurality of metric graphs, a metric graph of the plurality of metric graphs including first nodes corresponding to the vectors of outcomes, the first nodes being selectively connected based on a first criterion;
select, from the plurality of metric graphs and based on a second criterion, an optimal graph;
generate, based on the optimal graph, a clustered graph including second nodes corresponding to groups of the first nodes, the second nodes being selectively connected based on a third criteria;
generate a first layout of the clustered graph, the first layout including a two-dimensional (2D) representation of the clustered graph;
generate, partially based on the first layout, a second layout of the optimal graph, the second layout including a 2D representation of the optimal graph; and
display the second layout.
